(19) United States
(12) Patent Application Publication
(10) Pub. No.: US 2002/0188471 A1
(43) Pub. Date: Dec. 12, 2002

(54) Method to systematically integrate ancillary non-pharmacological therapy programs into research studies of pharmaceutical drugs and medical devices

(76) Inventors: Margaret S. Schaefer, Plainsboro, NJ (US); Patricia Flynn, Somerset, NJ (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/136,740

(22) Filed: Apr. 30, 2002

Related U.S. Application Data
(60) Provisional application No. 60/287,931, filed on Apr. 30, 2001.

(51) Int. Cl. G06F 17/60; G06F 19/00; G01N 33/48; G01N 33/50

(52) U.S. Cl. 705/2; 702/19

(57) ABSTRACT

A method for conducting research studies addresses behavioral aspects of a study participant's disease that affect a study participant's quality of life. Those aspects may be symptomatic of the disease itself or a side effect of treatment of an underlying disease. The research studies are conducted by adding a structured behavioral regimen to all study participants enlisted in a research study for testing a pharmacological drug or device.

### TECHNICAL FIELD

The present invention relates generally to systems and methods for conducting research studies. More particularly, the invention is directed to integrating ancillary non-pharmacological therapy programs into research studies of pharmaceutical drugs and medical devices.

### BACKGROUND OF THE INVENTION

Drug development and testing involves enormous resources to test such drugs for safety and efficacy. In just one example of the importance of drug studies, the Food and Drug Administration (FDA) require pharmaceutical companies to prove drug safety and efficacy before such drugs are granted an “FDA approval for sale”. Pharmaceutical companies must prove safety and efficacy through a “new drug approval” (NDA) process that includes research studies.

Of course, there are instances other than FDA related processes where research studies are important. Research studies may include randomized clinical trials, post-marketing and seeding studies typically sponsored by pharmaceutical, medical device and diagnostic companies. Other studies include pharmco-economic studies and consumer research studies of new over-the-counter products (e.g., skin care and nutritional product studies).

One particular kind of research study involves clinical trials. Clinical trials are studies conducted in a continuum of 4 phases to prove (1) safety and (2) efficiency: Preclinical and phases I-III.

The initial clinical trial phase is preclinical. Product development begins with the identification of promising compounds and concepts that are scientifically challenging and may fill unmet medical needs. Extensive testing is then done in laboratory settings to assess the usefulness of the candidate therapies and to ensure that these novel approaches are safe to administer in humans. The resulting scientific data are then compiled and a formal request made to the FDA for permission to advance to the next phase of research and to administer the new drug to study participants. This request is called an Investigational New Drug application, or an IND. Approval of the IND is given only after the scientific and ethical merits of the supporting research and the proposed phase I study have been evaluated in depth and experts concur with the company's recommendation to move forward.

Phase I studies are principally designed to examine the safety of a new medication and to begin to understand how the drug will work in humans through the gathering of extensive information to evaluate how the human body responds. Observations of how the medication is absorbed, distributed, metabolized and eliminated from the body are often made, along with assessments of how quickly a therapeutic concentration is achieved, how long the drug remains in the body, and what, if any, the effect drug metabolite by-products may have. With step-by-step increases in dose, the optimal dosage is eventually determined where minimum side effects are coupled with maximum therapeutic effect, termed the toxic-therapeutic window.

Many phase I studies enroll only healthy participants to evaluate how a new drug behaves in humans. In some instances, the FDA and investigating physicians deem it more appropriate for phase I trials to enroll study participants who suffer from the same disease that the new drug seeks to treat, rather than to study healthy volunteers. Candidates are enrolled in a study only after a review of their history and physical confirms their eligibility and an informed consent for treatment is given.

The number of participants enrolled in a phase I trial will vary depending on the stepwise progression established for achieving optimal dosing as well as prior clinical experience with similar compounds and approaches. As well, the FDA will recommend the required follow-up period for each study participant. Follow-up time periods may range from just a few days to six or more months. Further trials may continue only if phase I results indicate that the new therapy is reasonably safe in humans, and the FDA approves further investigations.

Phase II studies are principally designed to evaluate the therapeutic effect of a new drug in study participants who suffer from the targeted disease, and to confirm the safety profile established in earlier phase I trials. Second phase studies are sometimes placebo-controlled and often double-blinded where neither the study participant nor the medical personnel know if a placebo or the medication is being prescribed. Phase II trials tend to enroll a larger number of participants than in phase I, and study participant follow-up may be for longer periods. Phase II studies are tailored to specific treatment indications for which the company plans to seek broader approval. Phase II trials set the stage, and further establish parameters, for the longer-term phase III trials.

Recent reforms have been made to FDA procedures for clearing new drugs aimed at treating fatal diseases such as AIDS and cancer. In selected circumstances, and where compelling scientific evidence is presented, the FDA has indicated that it will expedite review of a company's application for market clearance. Expedited review of phase II clinical data, and clearance of that early application, can obviate requirements for phase III trials.

Phase III trials are principally designed to demonstrate the potential advantages of the new therapy over other therapies that are already on the market. Safety and efficacy of the new therapy are studied over a longer period of time and in many more study participants enrolled into the study with less restrictive eligibility criteria. The scope of phase III studies is intended to help scientists identify rarer side effects of treatment and to prepare for a broader application of the product.

Clinical trials are merely one example of the importance of conducting research studies on drugs. Such studies are relevant to develop new drugs as well as to test know drugs for new applications. As the above description summarizes, research studies are generally focused on the administration, safety, and efficacy of pharmaceutical drugs. However, many diseases may have a behavioral component such as smoking, obesity, and heart disease or may have a side effect that could be affected by a study participant's behavior. For example, a diabetes drug may have a side effect that causes weight gain. Providing research studies that also address underlying study participant lifestyle may increase study participant retention and provide more rigorous study results.

### SUMMARY OF THE INVENTION

The invention contemplates a system for conducting research studies that addresses behavioral aspects of a study participant's disease that affects a study participant's quality of life. Those behavioral aspects may be symptomatic of the disease itself or a side effect of treatment of an underlying disease. For example, weight gain is symptomatic of obesity and may be a side effect of medications used to treat diabetes. The research studies are conducted by adding a structured behavioral regimen to all study participants enlisted in a research study. As a result, study participant retention and compliance in the research study may be enhanced. By increasing study participant retention, fewer participants may need to be enrolled in a study because statistically required numbers of study participants will remain for the duration of the study. Hence, over enrollment to account for potential study participant losses over the life of the research study may be reduced, lowering the overall study costs. These costs along with more rigorous results may offset the increased per study participant cost required to administer the behavior program.

In accordance with the invention, drug study participants are enlisted from a population having a predetermined medical profile indicative of a predefined medical condition or disease. The study participants are randomly divided into different study participant sets. Each set is provided with a pharmacological treatment regimen that comprises a pharmacological component that may be a drug, topical ointment, medical device, etc. One of the sets of participants receive a pharmacological component that has been selected for testing in the treatment of the predefined medical condition. Other sets of study participants receive a placebo or a comparison drug (typically one that is known to treat the predefined medical condition). All study participants participating in the research study receive a behavioral modification regimen that is selected to treat behavioral components of the medical condition.

### BRIEF DESCRIPTION OF THE DRAWINGS

The foregoing summary, as well as the following detailed description of the preferred embodiments, is better understood when read in conjunction with the appended drawings. For the purpose of illustrating the invention, there is shown in the drawings exemplary constructions of the invention; however, the invention is not limited to the specific methods and instrumentalities disclosed. In the drawings:

FIG. 1 is flowchart providing an overview of a research study incorporating aspects of the present invention;

FIG. 2A is a detailed prior art chart of a research study for two study drug doses and a placebo;

FIG. 2B is a detailed chart of a research study in accordance with the invention for two study drugs and a placebo;

FIGS. 3A and 3B represent an example research study protocol overview in accordance with the invention;

FIGS. 4A and 4B represent a second example research study protocol overview in accordance with the invention;

FIGS. 5A-5E are examples of non-pharmacological therapy program information provided to study participants; and

FIGS. 6A and 6B are examples of non-pharmacological therapy program information provided to counselors in the research study in accordance with the invention.

### DETAILED DESCRIPTION OF THE INVENTION

Overview

The present invention addresses the need for providing a research study that addresses a holistic approach to disease treatment. The invention employs behavioral science programs to enhance pharmaceutical research study processes. Behavioral science refers to any programs, services, and/or products that encourage people to change their behavior. These can include—but are not limited to—starting an exercise program, quitting smoking, complying with physician recommendations, eating fruits/vegetables, and many other changes in behavior.

For example, in planning for the introduction of a new obesity drug into a market that may be skeptical of obesity drugs based on past experiences with this category, an intensive weight reduction program was added as an integral component to the research study protocol. As part of this program, registered nurses and dieticians were trained how to effectively coach study participants on diet and exercise for 15-20 minutes during each protocol visit. Counselor training, counselor materials, and study participant handouts were developed, translated (into 24 languages), printed, delivered and quality assurance for the behavioral science protocol was provided. Directors of the research study valued this holistic approach to obesity as a method to improve study participant recruitment and retention, facilitate FDA approval, improve study outcomes, differentiate the new drug in the marketplace, and reduce malpractice litigation.

A second example is the introduction of a new medicated skin cream. A new medicated cream for skin cancer may require strict protocol adherence to remain effective. A protocol reminder campaign could be incorporated into the clinical protocol. A Lifestyle Inventory could be administered to determine the study participant's readiness to change, perceived benefits and barriers, and primary motivating factors to comply with the protocol. Targeted electronic reminder messages could be sent as a phone call, pager, and/or email at a time and frequency determined by the study participant. This program would also aid in improved study outcomes, study participant retention, and facilitate FDA approval.

Exemplary Operation

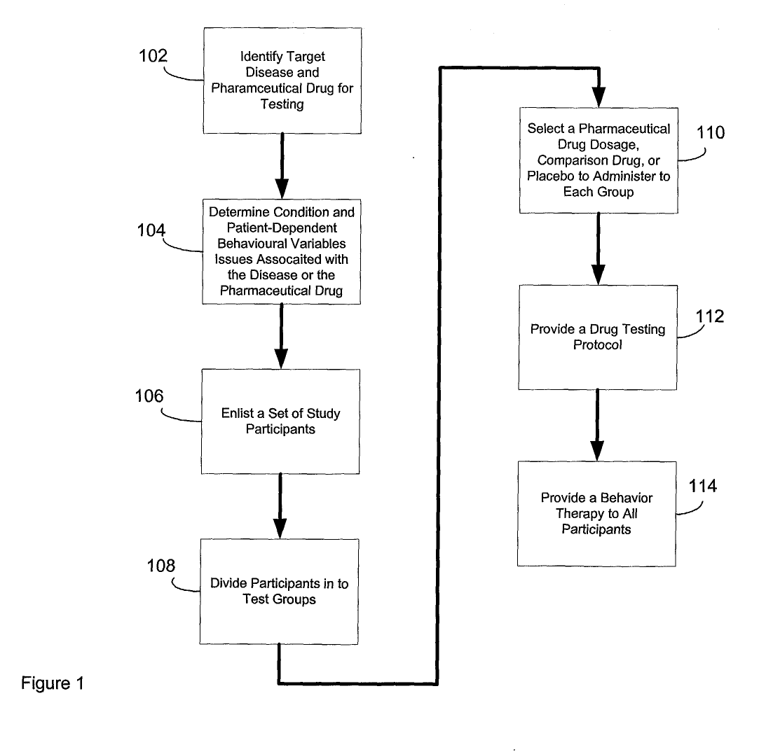

FIG. 1 is a flow chart of the process of a research study in accordance with the present invention. The first step (102) in the process is to identify a target disease to be treated and a pharmaceutical drug to be tested against the disease. For example, a target disease may be obesity and a study drug may be toprimate. After identifying the disease and drug, a research study is designed to test efficacy, safety, or both. For example, such a research study design may include various drug doses to be tested, the length of the study, and so on. One such study was a one-year long, randomized, double-blind, placebo-controlled study to assess the efficacy and safety of toprimate in the treatment of obese type 2 diabetic study participants that were being treated for diabetes with metformin. The study was designed to compare the efficacy and safety of 96 and 192 mg of toprimate daily with placebo.

In accordance with the invention, during the research study, aspects of the study participant's behavior is simultaneously treated with an ancillary non-pharmacological therapy program that is designed to provide behavior modification that address one or more behavioral conditions (step 104). The behavioral conditions may be contributing to the disease or may be a side effect of a pharmaceutical drug. For example, while toprimate was being tested as a treatment for obesity, it is know that diet and exercise programs have an effect on weight gain or loss. While behavioral components may be readily determined such as in the case of obesity, target populations may also be tested to determine quality of life issues in certain population segments. Such quality of life issues may be determined by way of surveys, SF-36, SF-8, public or private studies, and so on.

After the quality of life issues are determined, a counseling program is designed to address selected quality of life issues that can be mitigated through behavior modification. A behavior modification regimen is designed to address the selected issues, and the regimen is integrated into the research study protocol.

A set of drug study participants is enlisted to participate in the research study (step 106). Generally, the population could consist of a statistically significant number of male and female study participants that fall into a predetermined age group. One study for example comprised a randomized set of 570 male and female study participants between the ages of 18 and 75 years of age. The study participants were required to have a BMI between 27 and 50 kg/m², an established history of type 2 diabetes, have been on metformin monotherapy for at least four month with a stable dose for two months that did not exceed 2 g/day, HbA_{1c}≦9.5% at enrollment, FPG≧7 mmol/L (126 mg/dl) and <12 mmol/L (216 mg/dl) at enrollment, have has stable weight for three months, and not pregnant, planning a pregnancy, or breast feeding. Study participants in such study would be excluded in accordance with the study parameters. In the example toprimate study, study participants were excluded that had prior exposure to topiramate, had type 1 diabetes, were on antidiabetic agents other that metformin, experienced significant weight loss prior to entry and so on.

The eligible study participants are randomized into different study sets (step 108). Each set is given a different study drug regimen. One group may receive the pharmaceutical drug to be tested at a selected dosage, another may receive the same drug at a different dose, a third group may receive a placebo or a different pharmaceutical drug as a baseline comparison (step 110). As described more fully below, a study protocol is then provided that includes a pharmaceutical component (i.e., the drug dosage or placebo) and a behavioral regiment that is provided in conjunction with the study protocol (112-114).

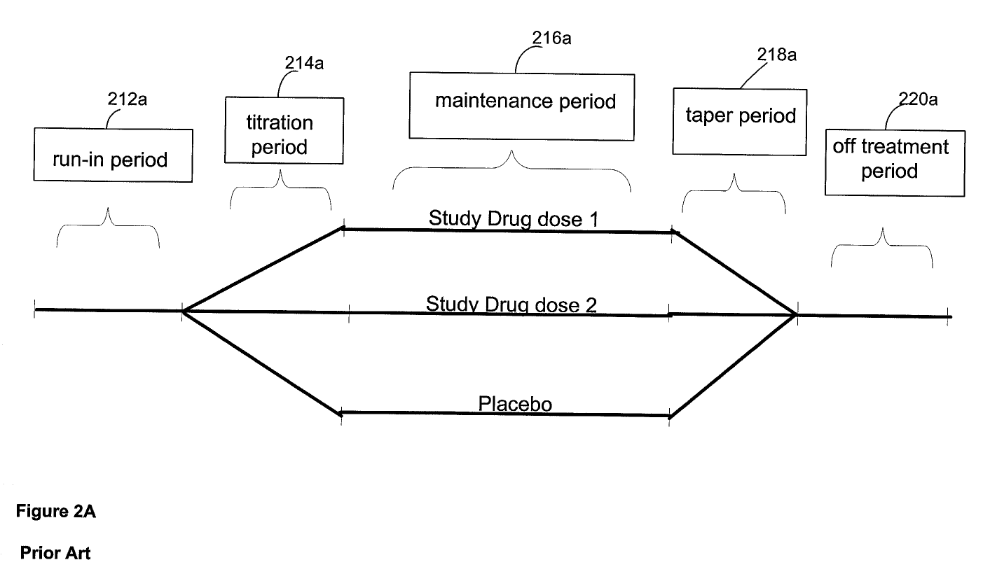

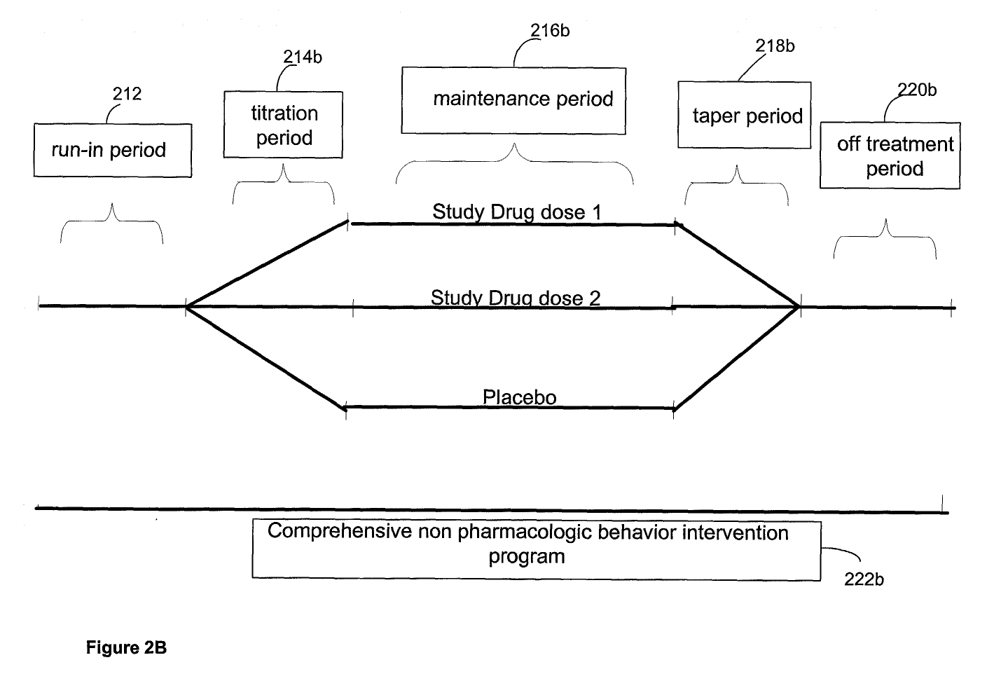

FIG. 2A graphically illustrates further detail of the stages of an exemplary research study. Here, the research study starts with a run-in stage 212a. During this stage the study participants are given thorough medical examinations, may be administered a small dose of the pharmaceutical drug and monitored for reactions, monitored for continued eligibility, and so on. After the run-in stage 212a, study participants begin a titration stage 214a. During the titration stage, drug dosages are generally increased to the study dosage, and monitored for acceptance of the drug, severe reactions to the dosage levels, etc. This is followed by the maintenance stage 216a, where the study participant is maintained on the selected dosage for a predetermined period. Thereafter, taper stage 218a begins wherein the drug dosage is slowly decreased. Finally, the follow-up stage 220a monitors adverse reactions, efficacy, safety, side effects, etc.

FIG. 2B is a graphical illustrates the stages of a research study such as the research study of FIG. 2a wherein a comprehensive non-pharmacological behavior intervention program has been provided along with the pharmaceutical protocol. Here, during all of the stages of the drug protocol 212b, 214b, 216b, 218b, 220b, the behavioral regimen 222b is simultaneously provided.

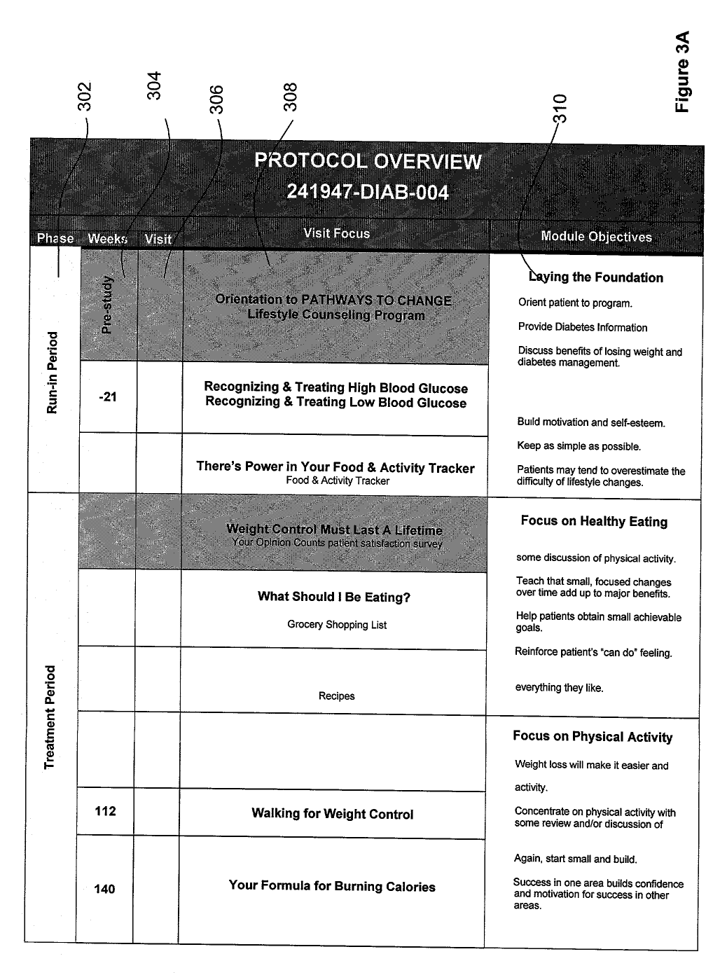

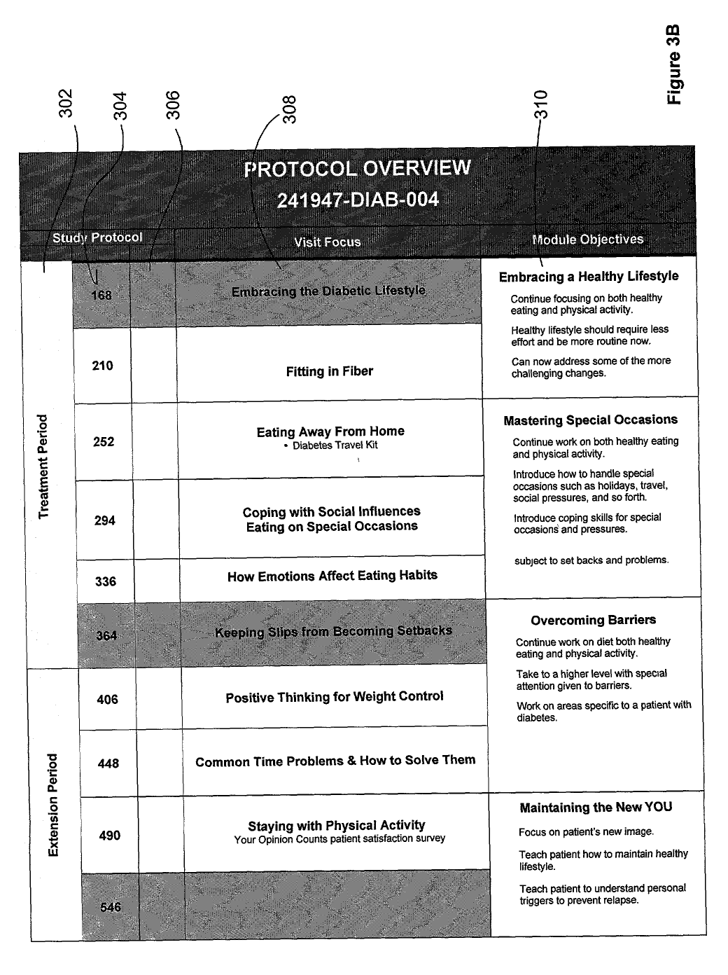

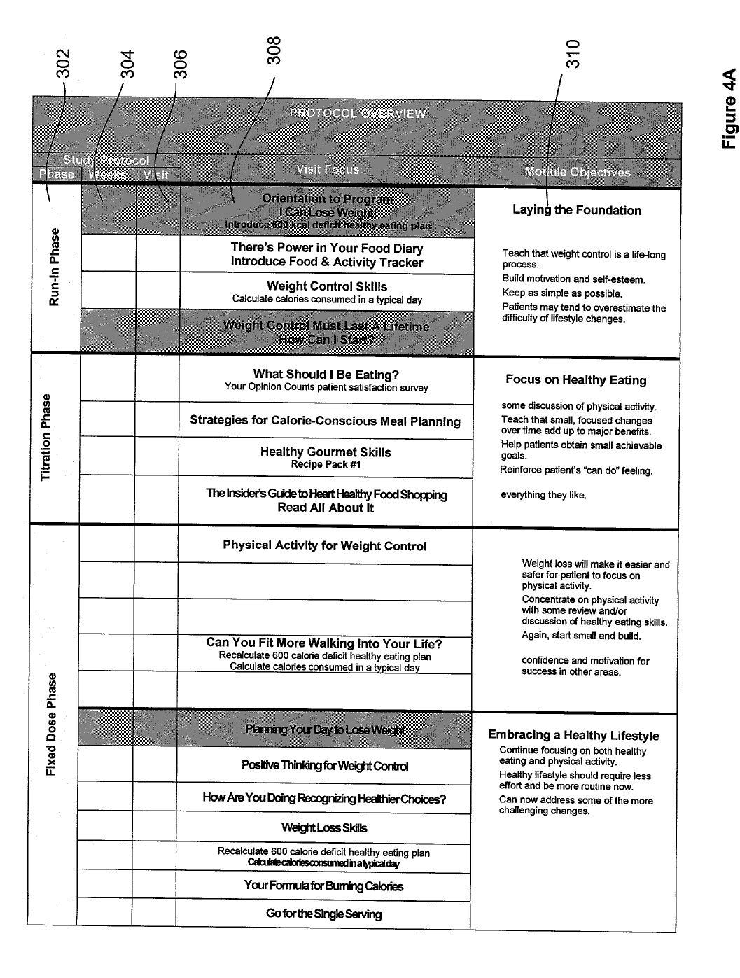

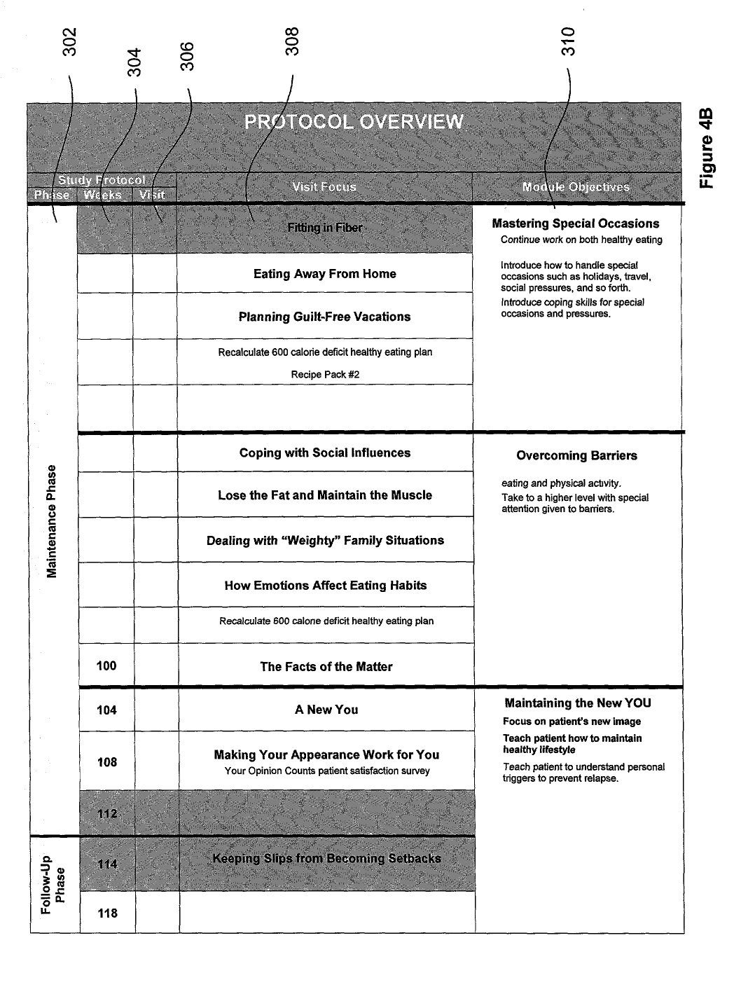

FIGS. 3A and 3B provide an example behavioral protocol overview that outlines a behavioral modification regimen that is provided to correspond to the stages of a pharmacologic protocol such as the protocol described in reference to FIG. 2B. In the protocol overview of FIGS. 3A and 3B, the clinical trail phases (or stages) are listed in column 302, the corresponding research study weeks are listed in column 304, the visit number (i.e. the study participants visit to the clinic) are listed in column 306. Columns 308 and 310 provide further details about the behavioral modification regimen provided during each visit. On each visit, a counselor provides all study participants with the same material. This provision is independent of the clinic to which the study participant is assigned. Here, the particular regimen is directed to weight control as a side effect of diabetes control. FIGS. 4A and 4B provide a comparable behavior modification protocol for use in conjunction with the pharmacologic treatment of obesity. Although the behavior modification regimens are described in reference to weight control, similar programs could be designed to treat other diseases and side effects that may have a large behavioral component such as smoking, heart disease, high cholesterol, high blood pressure, depression, or to address study participant compliance with drugs that require long periods of compliance to produce effects such as skin treatments for cancer or aging.

Appendix I provides a set of interview questions that are presented to study participants or information that is gathered by counselors during various stages (i.e. Phases) of the research study. The stages in Appendix I generally conform to the stages outlined in FIG. 2B.

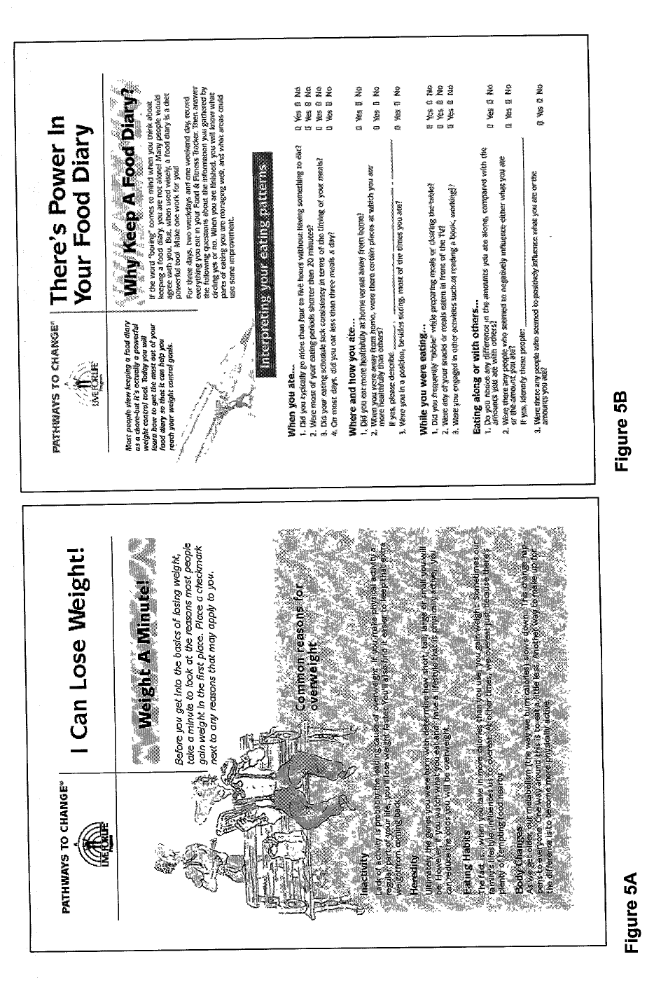

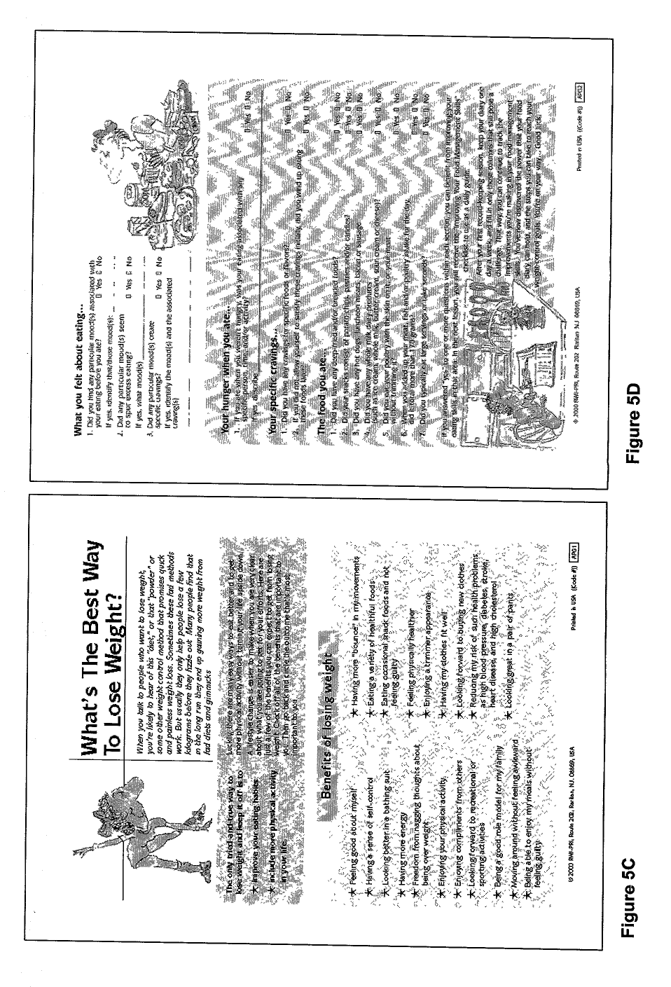

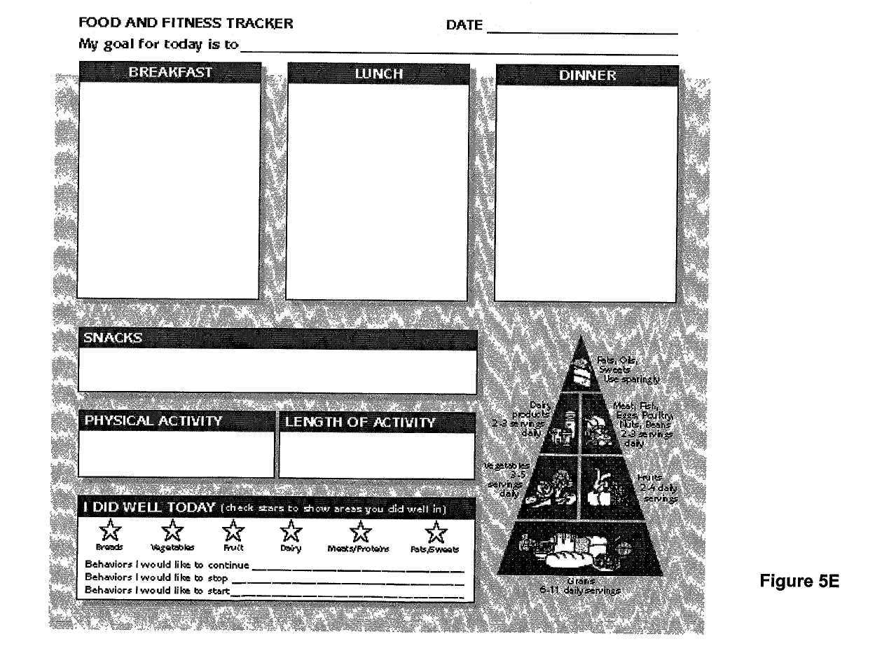

FIGS. 5A through 5D illustrate an exemplary set of materials that are provide to study participants to aid in the education and feedback process of the behavioral modification program. For example, FIG. 5A provides education information about the causes of weight gain. FIG. 5B is a questionnaire that helps study participants identify their eating patterns and provides feedback for behavioral modification. FIG. 5C provides education material about how to lose weight and some of the benefits of losing weight. FIG. 5D is a questionnaire that provides feedback to help study participants understand their eating patterns. FIG. 5E is an example of a diary page that study participants use to track their daily eating and activity patterns.

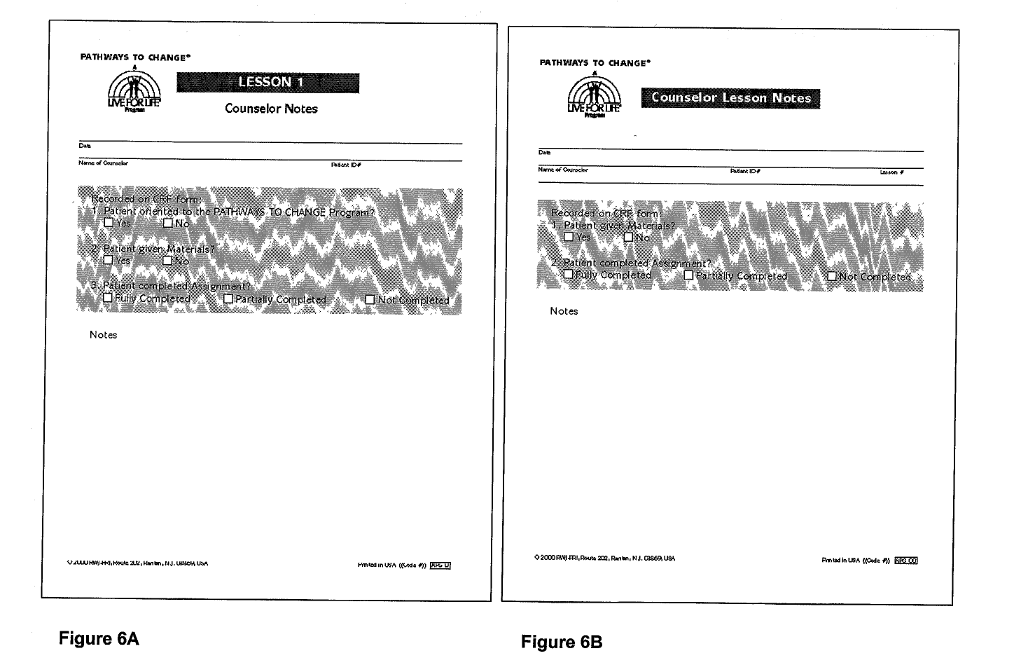

According to an aspect of the invention, the behavior modification regimen is standardized, to the extent possible, for all study participants participating in the study. This is irrespective of which particular clinic is administering the protocol. Accordingly, standard materials and training information is provided to counselors (e.g., doctors, nurses, etc.) to ensure that the program is properly administered. FIGS. 6A and 6B are exemplary materials provided to counselors to track each lesson to ensure a degree of uniformity. Here, the materials are in the form of checklists to ensure that the study participant has complied with the prescribed behavioral regimen of lesson 1 of the protocol.

For pharmaceutical research studies, it is believed that such a behavioral modification regimen can help to improve study participant recruitment and retention, differentiate the product from competition, enhance product effectiveness, facilitate the FDA approval process, and avoid/reduce malpractice litigation. Moreover, study participants receiving placebo treatment would also benefit from the changed behavior. However, the program runs the risk of confounding the effects of the research study when looking at two drugs (versus a drug and a placebo). That is, the behavioral modification could cause the effects of the drugs to be masked in the results (as opposed to the results from a completely random group of study participants that were not encouraged to change their behavior). Additionally, the cost associated with administering the program would drive up the cost per study participant. However, these negative are believe to be offset by the more rigorous research study results as well as reduced overall costs resulting from study participant retention.

[image]

[image]

[image]

[image]

[image]

[image]

[image]

What is claimed is:

1. A method for conducting pharmacological research studies, comprising:
enlisting a set of study participants from a population wherein the enlisted participants meet a predetermined medical profile indicative of a predefined medical condition;
dividing the study participants into a first set of participants and a second set of participants;
providing a pharmacological treatment regimen comprising a pharmacological component; exposing the first set of participants to the pharmacological treatment regimen, wherein said pharmacological component comprises a component selected for testing in the treatment of the predefined medical condition;
exposing the second set of participants to the pharmacological treatment regimen, wherein said pharmacological component comprises a second effect upon the medical condition; and
providing a behavioral modification regimen selected to treat behavioral components of the medical condition; and
exposing the first and second group to the behavioral modification regimen.

2. The method as recited in claim 1 further comprising assigning the study participants to one of a plurality of clinics.

3. The method as recited in claim 1 wherein the second effect upon the medical condition is a placebo effect selected to have no pharmacological effect upon the medical condition.

4. The method as recited in claim 1 wherein the component selected for testing comprises a pharmaceutical drug.

5. The method as recited in claim 1 wherein the component selected for testing comprises a medical device.

6. The method as recited in claim 1 wherein the predefined medical condition is a disease.

7. The method as recited in claim 1 wherein the predefined medical condition is a side effect of a pharmaceutical drug.

8. The method as recited in claim 6 wherein the disease comprises a disease selected from the group comprising obesity, high blood pressure, smoking, high cholesterol, and depression.

9. The method as recited in claim 7 wherein the side effect comprises a side effect selected from the group comprising weight gain, high blood pressure, smoking, high cholesterol, and depression.

10. The method as recited in claim 1 wherein the behavioral modification regimen comprises providing a prescribed counseling program for each visit to a clinic.

11. A method for conducting research studies, comprising:
enlisting a set of study participants from a population wherein the enlisted participants meet a predetermined medical profile and have a predefined medical condition;
randomly dividing the study participants into a first set of participants and a second set of participants;
providing the first set of participants a first treatment regimen said first treatment regimen selected for testing its effect on the predefined medical condition;
providing the second set of participants a second treatment regimen selected to have no pharmacological effect on the predefined medical condition; and
providing the first set of participants and the second set of participants with a behavioral modification regimen selected to treat behavioral components of the predefined medical condition.

12. The method as recited in claim 11 further comprising assigning the study participants to one of a plurality of clinics.

13. The method as recited in claim 11 wherein the first treatment regimen comprises a pharmaceutical drug.

14. The method as recited in claim 12 wherein the first treatment regimen comprises a medical device.

15. The method as recited in claim 11 wherein the predefined medical condition is a disease.

16. The method as recited in claim 11 wherein the predefined medical condition is a side effect of a pharmaceutical drug.

17. The method as recited in claim 15 wherein the disease comprises a disease selected from the group comprising obesity, high blood pressure, smoking, high cholesterol, and depression.

18. The method as recited in claim 16 wherein the side effect comprises a side effect selected from the group comprising weight gain, high blood pressure, smoking, high cholesterol, and depression.

19. The method as recited in claim 1 wherein the behavioral modification regimen comprises providing a prescribed counseling program for each visit to a clinic.

20. The method as recited in claim 19 wherein the behavioral modification regimen comprises providing a prescribed diet to each study participant.
